(19) United States
(12) Patent Application Publication
(10) Pub. No.: US 2006/0058189 A1
(43) Pub. Date: Mar. 16, 2006

(54) Ftsz-polypeptides as a target for herbicidal compounds

(76) Inventors: Ralf Reski, Oberried (DE); Eva Decker, Freiburg (DE); Justine Kiessling, Lauingen a.d. Donau (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/538,530

(22) PCT Filed: Dec. 12, 2003
(86) PCT No.: PCT/EP03/14162

§ 371 Date: Jun. 10, 2005

Related U.S. Application Data
(63) Continuation-in-part of application No. 60/433,556, filed on Dec. 13, 2002.

(51) Int. Cl. A01N 25/00; C07H 21/04; C12N 15/82; C12N 15/87; C12N 5/04; A01H 1/00; C07K 14/415

(52) U.S. Cl. 504/116.1; 530/370; 435/468; 435/419; 800/278; 536/23.6

(57) ABSTRACT

The present invention relates to nucleic acids containing a nucleotide sequence encoding a polypeptide as a target for herbicidal compounds, to said polypeptides or biologically active derivatives thereof, and to a mehotd for developing herbicidal compounds using test systems containing said polypeptides.

The present invention relates to nucleic acids containing a nucleotide sequence encoding a polypeptide as a target for herbicidal compounds, to said polypeptides or biologically active derivatives thereof, and to a method for developing herbicidal compounds using test systems containing said polypeptides.

In prokaryotes, cell division is mediated by the protein FtsZ which assembles into a ring structure at the future site of cytokinesis. As tubulin and FtsZ share similar three-dimensional structures and polymerize to related structures in vitro, FtsZ is supposed to be the ancestor of tubulin. In general, only one FtsZ gene is found in all recent eubacteria. Plastids as eukaryotic organelles of cyanobacterial origin have inherited the conserved mechanism of division by FtsZ proteins. Here, the corresponding gene was transferred to the nucleus during establishment of endosymbiosis and the encoded proteins are translocated into the plastids via signal peptides preceding the mature protein. In contrast to prokaryotes, plants harbor several nuclear-encoded FtsZ homologs indicating functional diversity of these proteins (R. Reski, Trends Plant Sci. 7, 103 (2002)). In phylogenetic analyses all FtsZ proteins of a given plant species cluster in two distinct families, FtsZ1 and FtsZ2. All plant FtsZ proteins analyzed so far were exclusively targeted to plastids.

Due to the economical and ecological impact of herbicidal compounds in agriculture, their development and mode of action are of great importance.

Thus, the technical problem underlying the present invention is to provide new target systems for herbicidal compounds, which can be used for developing new compounds having herbicidal activity.

The solution to the above technical problem is achieved by providing the embodiments characterized in the claims. In particular, there is provided a nucleic acid containing a nucleotide sequence encoding a polypeptide having an amino acid sequence selected from the group consisting of SEQ ID No. 3, SEQ ID No. 4 and SEQ ID No. 5 or a biologically active derivative thereof, said polypeptide or biologically active derivative thereof being a target for herbicidal compounds in plants. In a preferred embodiment of the present invention, the above defined nucleotide sequence is selected from the group consisting of SEQ ID No. 1 and SEQ ID No. 2.

The term “derivative” means a proteinaceous compound comprising a substitution, an addition, an insertion and/or deletion of one or more amino acid(s) in comparison to the amino acid sequences depicted in SEQ ID No. 3, SEQ ID No. 4 or SEQ ID No. 5. The term “biologically active” means that the derivative can be used at least as a target for herbicidal compounds in plants as is the case for the above-defined polypeptide.

In a further embodiment, the present invention relates to vector containing the above-defined nucleic acid. The term “vector” refers to a DNA and/or RNA replicon that can be used for the amplification and/or expression of the above defined nucleotide sequence. The vector may contain any useful control unit such as promotors, enhancers, or other stretches of sequence within the 5′ and/or 3′ regions of the nucleic acid serving for the control of its expression. The vector may additionally contain sequences within the 5′ and/or 3′ region of the nucleotide sequence, that encode amino acid sequences which are useful for the detection and/or isolation of the protein which may be encoded by the nucleotide sequence. Preferably, the vector contains further elements that enable the stable integration of the above-defined nucleic acids into the genetic material of a host organism and/or the transient expression of the nucleotide sequence of the above-defined nucleic acids. It is also preferred to use vectors containing selectable marker genes which can be easily selected for transformed cells. The necessary operations are well-known to the person skilled in the art.

A further embodiment of the present invention relates to a host organism containing the above-defined nucleic acid or the above-defined vector. Examples of suitable host organisms include various eukaryotic and prokaryotic cells, such as E. coli, insect cells, plant cells, mammalian cells such as CHO cells, and fungi such as yeast. In a preferred embodiment of the present invention, the host organism is a transgenic plant containing the above-defined nucleic acid or the above-defined vector.

A further embodiment of the present invention relates to a polypeptide having an amino acid sequence selected from the group consisting of SEQ ID No. 3, SEQ ID No. 4 and SEQ ID No. 5 or a biologically active derivative thereof. The polypeptide or biologically active derivative thereof are used as a target for herbicidal compounds. In a preferred embodiment of the present invention, the polypeptide or biologically active derivative thereof are localized in the cytosol of plant cells, and involved in cell division. Therefore, this embodiment is particularly suitable as a target for herbicidal compounds.

In a yet further embodiment of the present invention there is provided a method for developing herbicidal compounds, comprising the steps of:
contacting a test system containing the above-defined polypeptide or biologically active derivative thereof, with a candidate compound to be assayed; and
measuring the herbicidal activity of said candidate compound.

The test systems used in the method according to the present invention, may be, for example, the above-defined polypeptides or biologically active derivatives thereof per se, such as in solution or immobilized on a substrate, e.g. in form of a biochip, or the above-defined host organism. The measuring of the herbicidal activity of the candidate compounds to be assayed can be performed with any methods known in the art. Using the above-defined method of the present invention new herbicidal compounds can be provided with improved mode of actions.

The figures show:

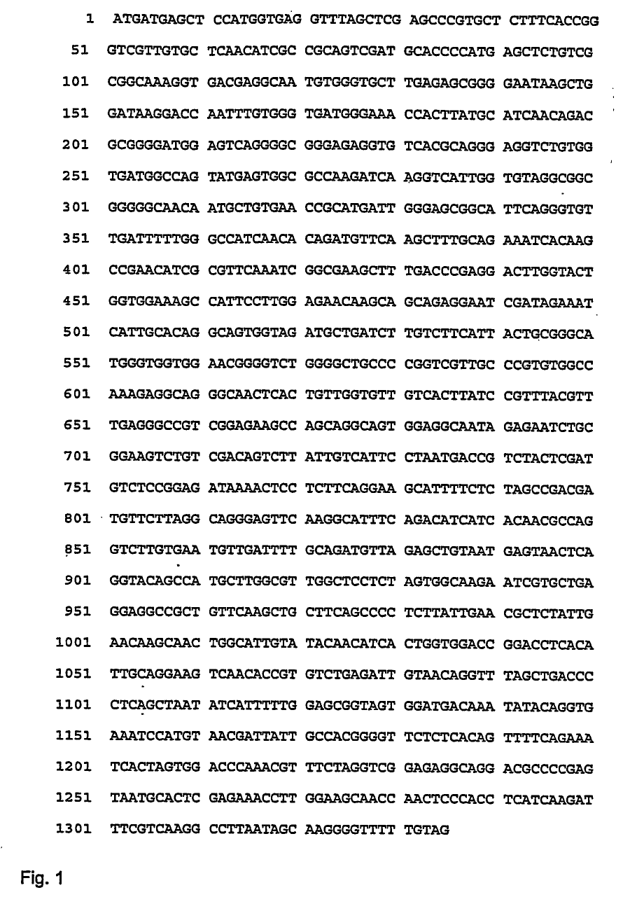

FIG. 1 depicts the nucleotide sequence of the ftsZ 1-1 gene (SEQ ID No. 1).

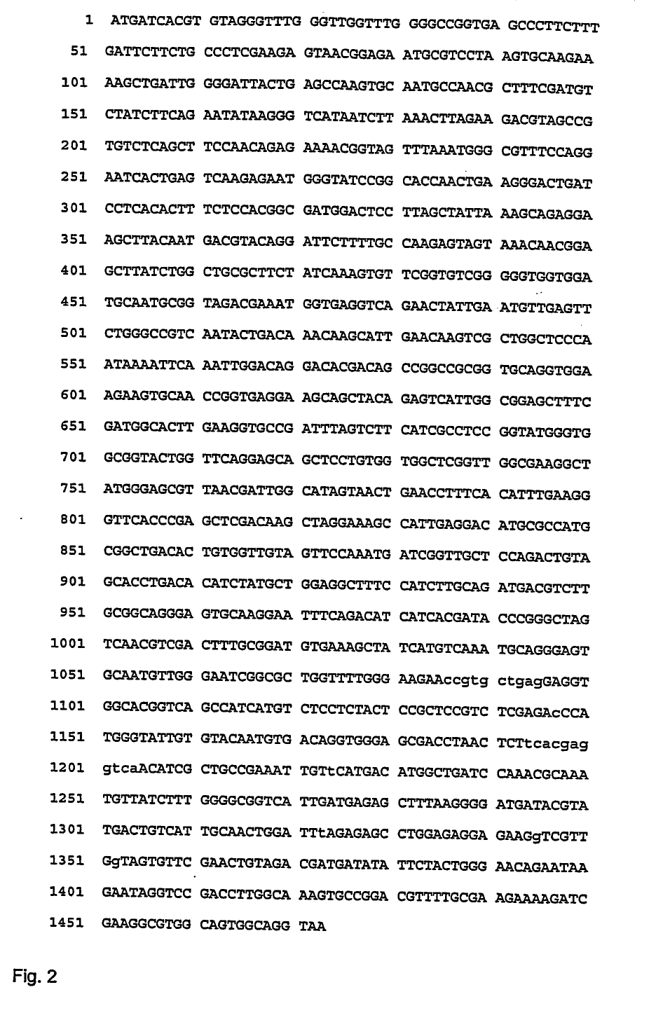

FIG. 2 depicts the nucleotide sequence of the ftsZ 1-2 gene (SEQ ID No.2).

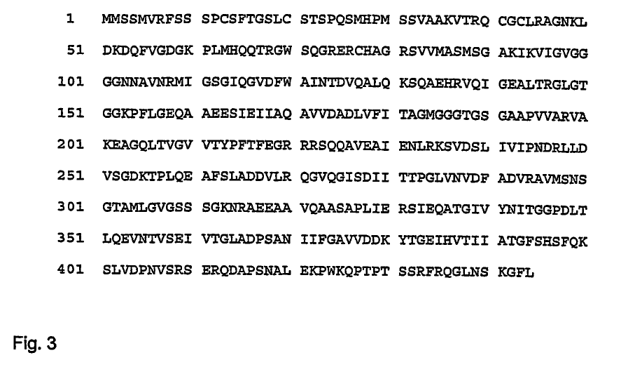

FIG. 3 depicts the amino acid sequence of ftsZ 1-1 (SEQ ID No. 3).

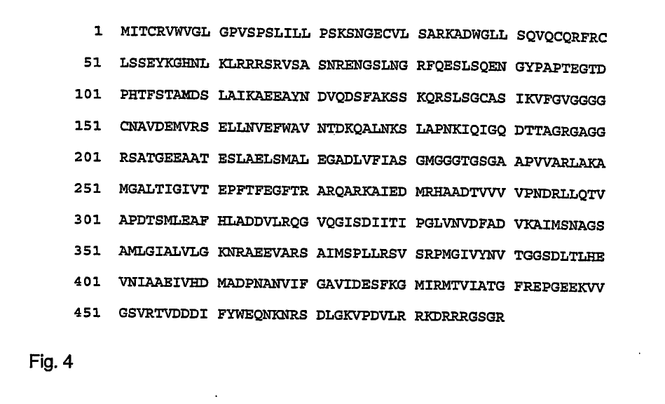

FIG. 4 is the amino acid sequence of FtsZ 1-2 (SEQ ID No. 4).

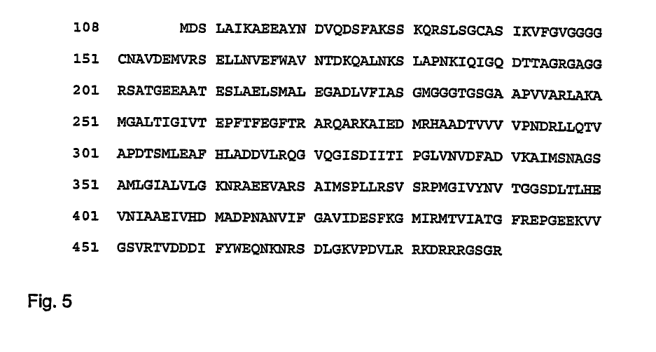

FIG. 5 is the amino acid sequence of truncated FtsZ 1-2 starting at position 108 (SEQ ID No. 5).

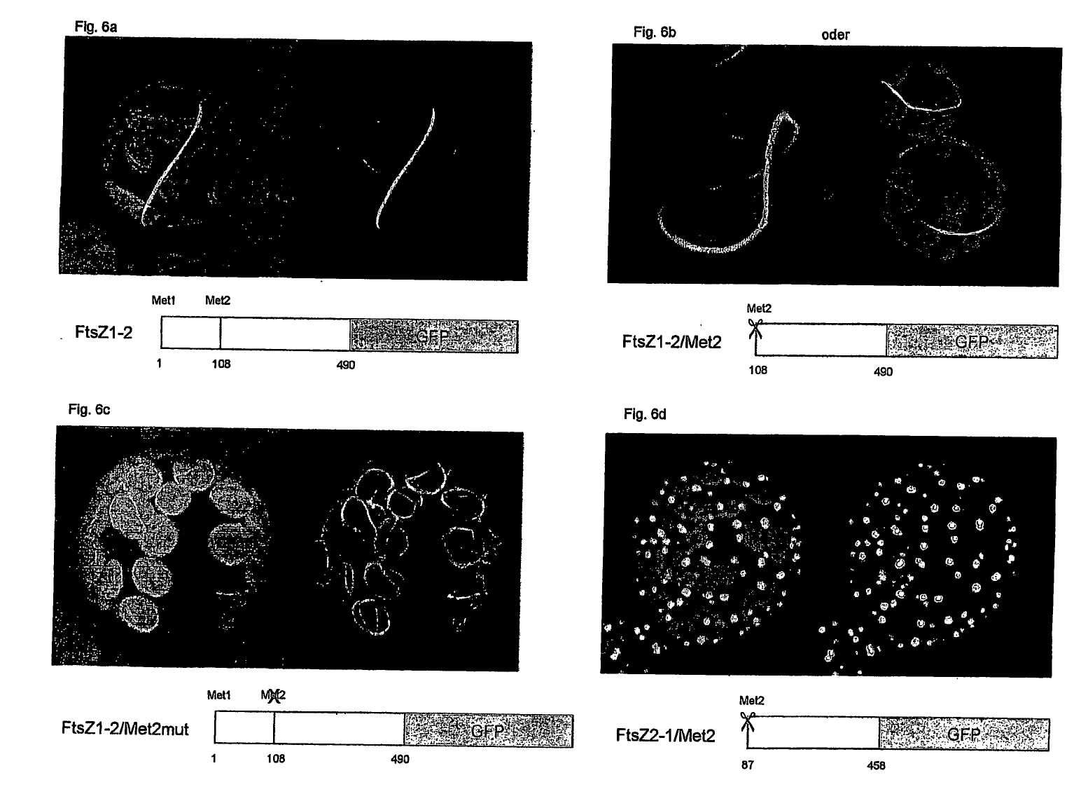

FIG. 6 illustrates the sub cellular localization of PpFtsZ::GFP fusion proteins. Physcomitrella protoplasts were transiently transfected with the corresponding expression vector and GFP fluorescence was visualized by confocal laser scanning microscopy 2 d after transfection (green: GFP, red: chlorophyll). (a) PpFtsZ1-2 full length (aa 1490)::GFP. (b) PpFtsZ1-2 without transit peptide (aa 108-490)::GFP. (c) PpFtsZ1-2 full length, methionine 108 mutated (aa 1-490)::GFP. (d) PpFtsZ2-1 without transit peptide (aa 87458)::GFP.

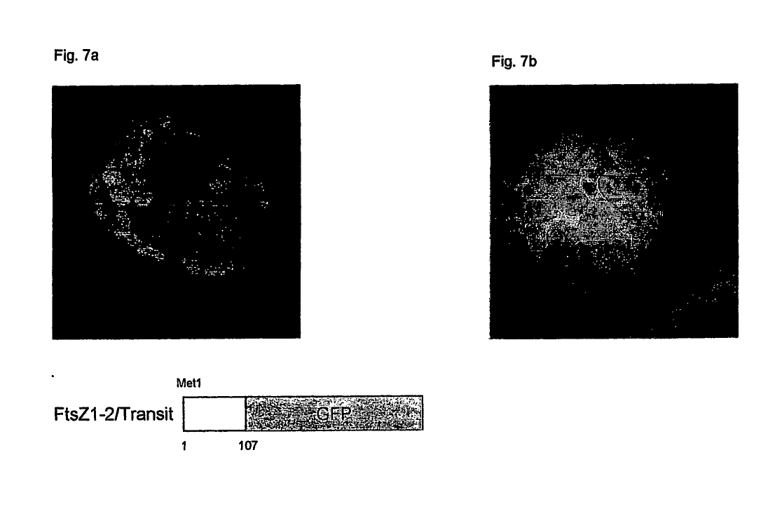

FIG. 7 illustrates the sub-cellular localization of FtsZ::GFP fusion proteins. (a) PpFtsZ1-2 transit peptide (aa 1-107)::GFP. (b) GFP in the cytosol of Physcomitrella patens.

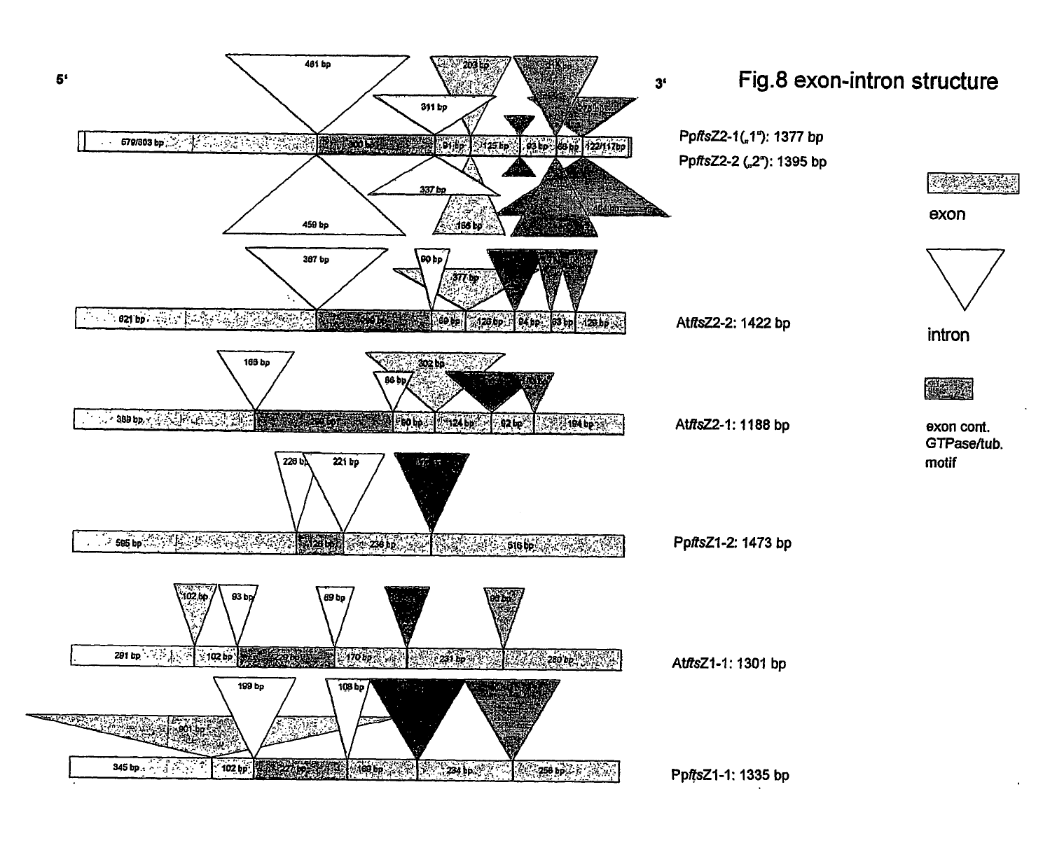

FIG. 8 illustrates the exon-intron structure of Physcomitrella and Arabidopsis FtsZ genes. Exons are shown as rectangular boxes, introns as triangles. The exon containing the GTPase/tubulin motif is shown in pink. Introns that are assumed to be homologous are presented in the same colour throughout the sequences.

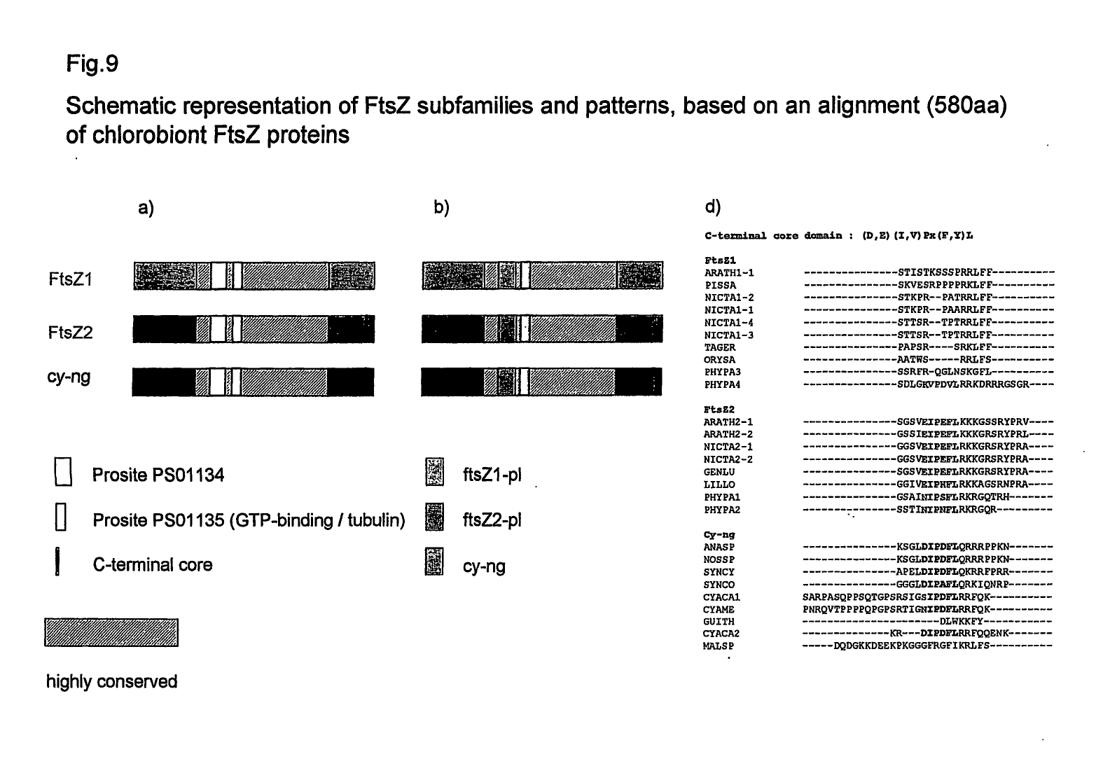

FIG. 9 is a schematic representation of FtsZ subfamilies and paftems, based on an alignment (580aa) of chlorobiont FtsZ proteins. a) The location of the two FtsZ PROSITE motives is shown as well as the C-terminal core domain and the highly conserved region of the protein together with b) the new motives. c) The two existing PROSITE FtsZ motives are shown as well as the detailed motives presented in this work. d) Presence or absence of the C-terminal core domain including mismatches.

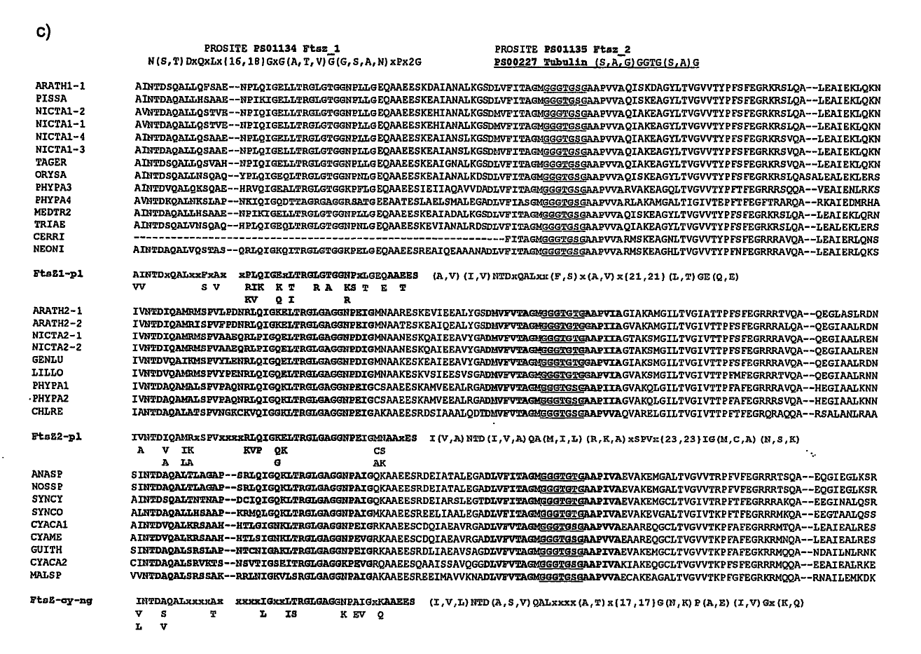

FIG. 10 illustrates that an anti-FtsZ antibody labels an annular-discoidal structure adjacent to the cell plate of dividing cells. (A) Western blot of Physcomitrella protein extract, probed with preimmune serum (left lane), anti-FtsZ1-2 (middle lane), and anti-tubulin (right lane), respectively. The signals at −40 and 46/47 kD represent the expected size of cytosolic and plastidic FtsZ1-2 (middle lane). Anti-tubulin detected proteins at −51 kD (right lane). (B-G) Immunocytochemistry using Physcomitrella protonema, a moss cell filament. Merge of FITC-signal (green, anti-FtsZ1-2) and chlorophyll (red). Anti-FtsZ detects an annular-discoidal structure adjacent to the cell plate (B-F) and the division site of plastids (G). (H,I) Double-labeling of FtsZ and tubulin displaying a microtubular preprophase band. (H) FtsZ-indicating FITC-signal, (I) tubulin detecting TexasRed-signal. Scale bars 5 μm.

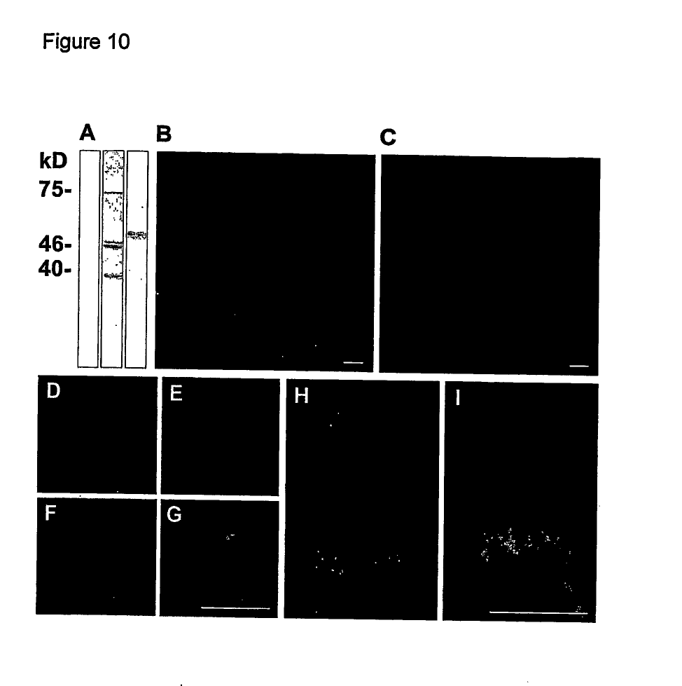

FIG. 11 illustrates that cytosolic FtsZ1-2 acts on eukaryotic cell division. (A) Physcomitrella cells that had been transiently transfected with different ftsZ-GFP expression plasmids were monitored for lack of division nine days after transfection. For construct description see FIG. 6a (dual targeting of full-length FtsZ1-2 to the cytosol and chloroplasts), FIG. 6b (targeting of truncated FtsZ1-2 to the cytosol), FIG. 6c (targeting of mutated FtsZ1-2 to chloroplasts), FIG. 6d (targeting of truncated FtsZ2-1 to the cytosol). Non-transfected cells served as control. Error bars represent the standard deviation from the mean of three independent transfection assays. (B, C) Cells twelve days after transient transfection with ftsZ1-2-GFP demonstrating a block in cell (B) and chloroplast division (C). Scale bars 10 μm

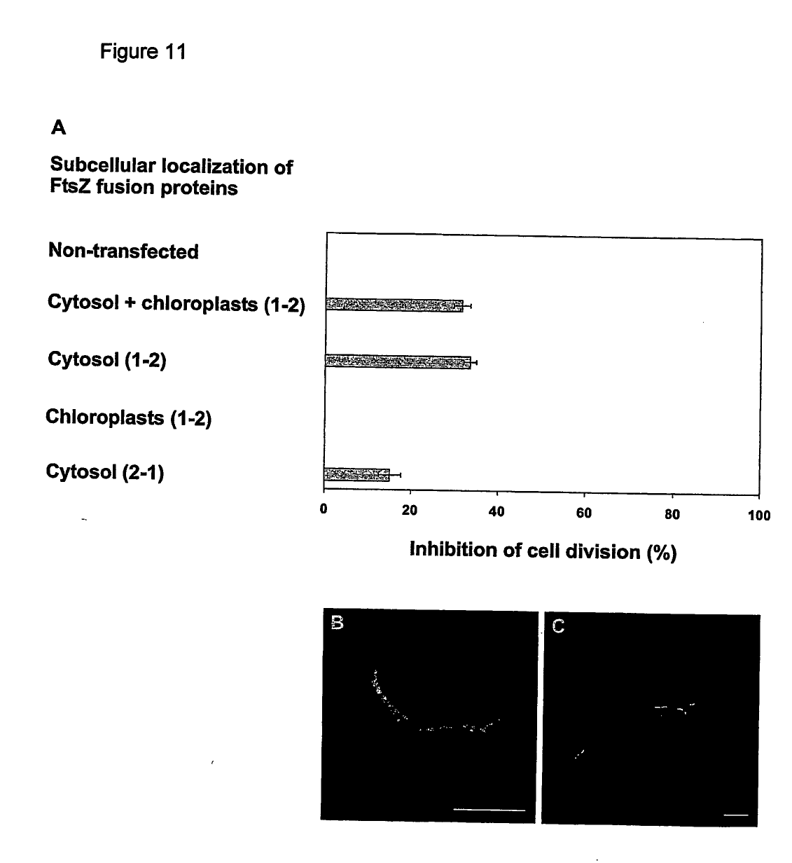

FIG. 12 illustrates that PpFtsZ1-1 is localized inside plastids. (A-D) Analysis of protoplasts transiently transfected with full-length FtsZ1-1-GFP demonstrated a localization within chloroplasts. In addition, the fusion protein seemed to connect chloroplasts via filaments (A, B, indicated by arrows). (C,D) Rarely, rings in addition to filamentous networks could be detected inside plastids. (E,F) Plastidic localization of PpFtsZ1-1 (aa 1-84)::GFP. (G,H) Artificial targeting of FtsZ1-1 to the cytosol via deletion of aa 1-84 (FtsZ1-1 (aa 85444)::GFP). (A,C,E,G) Merge of chlorophyll and GFP channel. (B,D,F,H) GFP channel only. Bars, 10 μm.

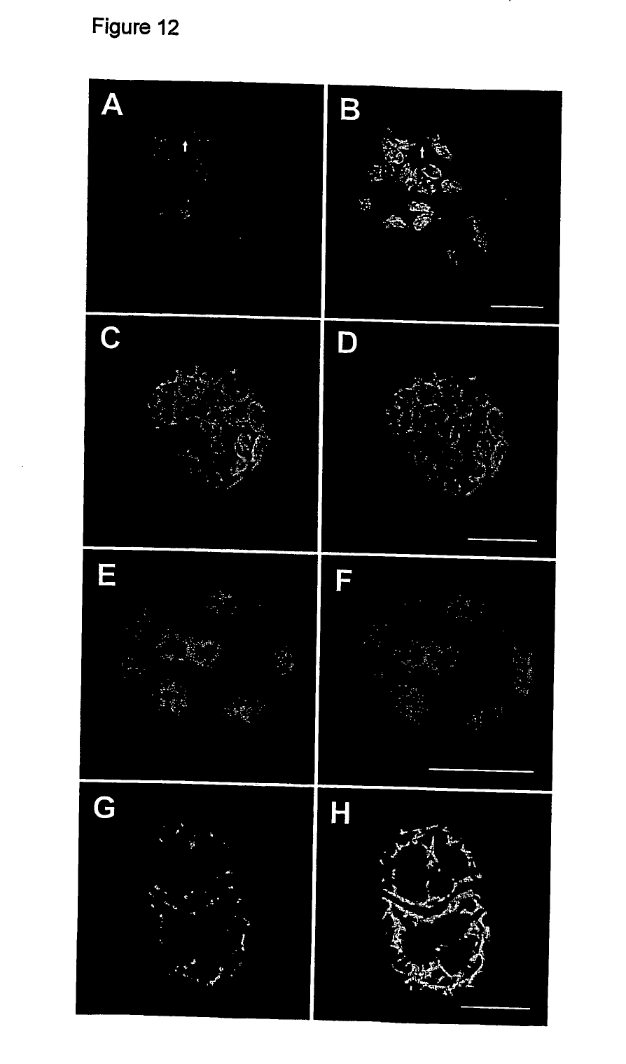

FIG. 13 shows that all PpFtsZ isoforms have dose-dependent effects on chloroplast division. Physcomitrella protoplasts transiently transfected with (A,B,E,F) PpftsZ1-1-gfp and (C,D) PpftsZ1-2 full length, methionine 108 mutated (aa 1490)::GFP (the second ATG was mutated to ATC resulting in a plastidic localization in contrast to the non-mutated FtsZ1-2 which is dual targeted to plastids and the cytoplasm). (A-D) Low levels of the corresponding fusion protein (indicated by a faint GFP fluorescence) resulted in “minichloroplasts” which are smaller in size compared to wild-type (for comparison with wild-type chloroplasts, refer to the bottom of (C)). (E,F) High levels of FtsZ1-1-GFP (indicated by strong GFP fluorescence) resulted in a block of plastid division leading to a single or a few huge plastids per cell. (A,C,E) Merge of GFP and chlorophyll channel. (B,D,F) GFP channel only. Bars, 10 μm.

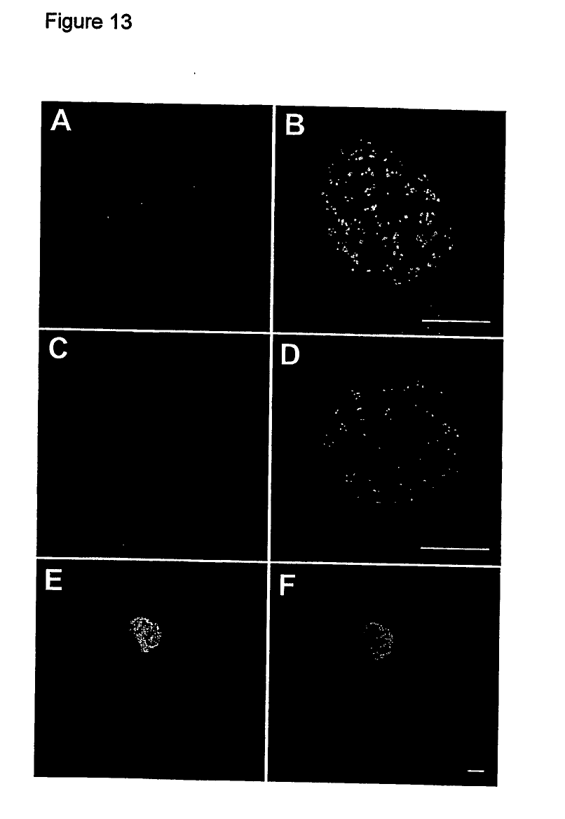

FIG. 14 illustrates acceptor photobleaching of Physcomitrella protoplasts transfected with different control constructs. Protoplasts were transfected with a cfp-yfp fusion (A), ftsZ1-2-cfp and ftsZ1-2-yfp (B,D,F-K,M,O), ftsZ2-1-cfp and ftsZ2-1-yfp (C,E,N,P), ftsZ1-2-cfp and yfpmcs (L). Using these protoplasts, FRET was analyzed via acceptor bleaching. (A,L) Spectra of an image point within a bleached region of a representative positive (A) and negative control (L). (B,C,M,N) Fluorescence intensities of CFP (blue) and YFP (yellow). (B,C) Bleaching was performed between time point 2 and 3 (ranging from a duration of 40-55 s). Before and after the bleach, CFP and YFP fluorescence was collected in approximately 16 seconds-intervals. Due to blocked FRET, CFP intensity increased after bleaching the acceptor. (M,N)CFP fluorescence did not increase in a non-bleached control area. (F-K) A representative cell (1-2a) expressing ftsZ1-2-cfp (F,I) and ftsZ1-2-yfp (G,J) was bleached in a defined region (red square) while a comparable non-bleached area (green square) served as control. (F-H)CFP (F), YFP (G) and the merge of both channels (H) before bleaching. (I-K) CFP (I), YFP (J) and the merge of both channels (K) after bleaching. (D,E,O,P) FRET efficiencies of representative cells (a,b) transfected with ftsZ1-2-cfp and ftsZ1-2-yfp (D,O) and ftsZ2-1-cfp and ftsZ2-1-yfp (E,P). High FRET efficiencies were obtained in bleached areas (D,E) whereas low or negative values were obtained in non-bleached control areas (O,P). Bar, 10 μm.

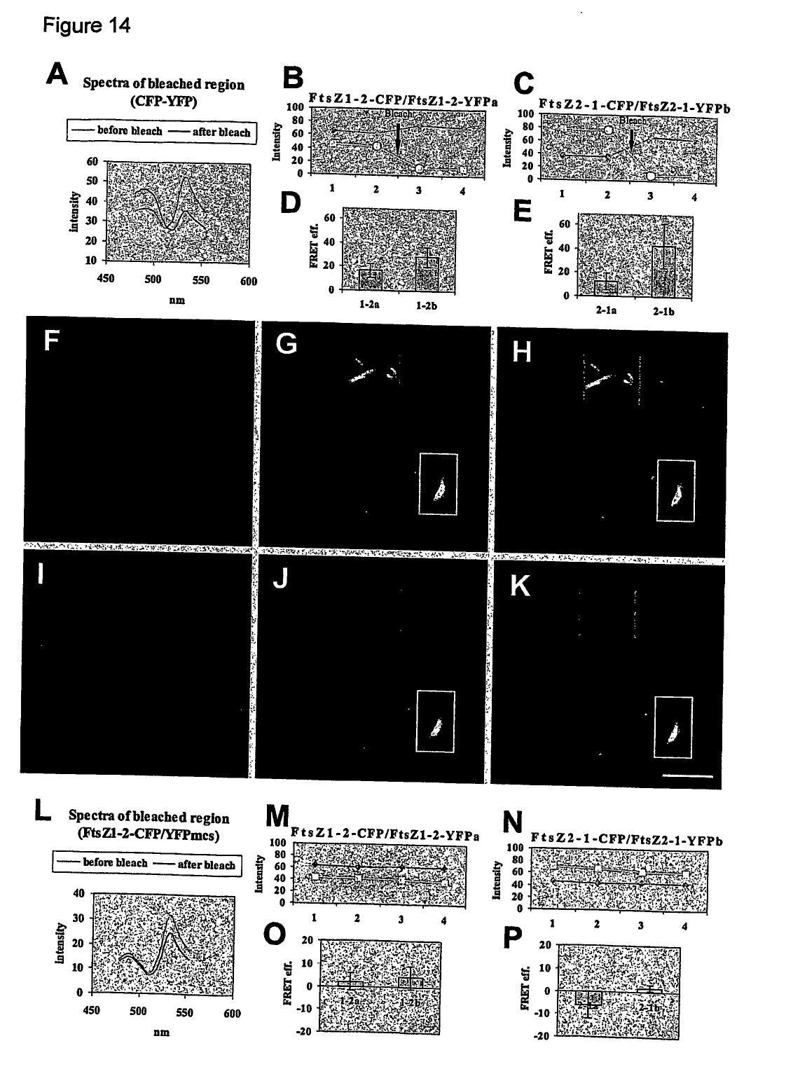

FIG. 15 shows the acceptor photobleaching of a protoplast transfected with ftsZ1-1-yfp and ftsZ2-1-cfp. A representative cell expressing ftsZ2-1-cfp (A,D) and ftsZ1-1-yfp (B,E) was bleached in a defined region (red square) while a comparable non-bleached area (green square) served as control. (A-C)CFP (A), YFP(B) and the merge of both channels (C) before bleaching. (D-F) CFP (D), YFP (E) and the merge of both channels (F) after bleaching. (G,I) Fluorescence intensities of CFP (blue) and YFP (yellow) showed an increase of CFP fluorescence after bleaching a defined area (G) thus revealing a direct protein-protein interaction of FtsZ1-1 and FtsZ2-1. In a non-bleached control region, CFP intensity slightly decreased due to the imaging process (I). (H,J) FRET efficiencies obtained from representative experiments (a,b): (H) bleached, (J) non-bleached regions. (K,L) Representative spectra of an image point within a bleached (K) and non-bleached (L) region of the cell shown in (A-F). Bar, 5 μm.

The following non-limiting examples further illustrate the present invention.

### EXAMPLES

### Example 1

### Characterization of ftsZ 1-1 and ftsz 1-2 and the Corresponding Polypeptides

Cloning and Sequencing

Two new Physcomitrella patens FtsZ genes are being presented in this study, FtsZ 1-1 (AJ428993) and 1-2 (AJ428994). From a clustered EST database covering nearly the whole transcriptome (Rensing et al., 2002), clusters defining the previously unknown sequences were revealed by homology searching with members of the plant FtsZ1/2 families. Subsequent cloning and sequencing as well as RACE-PCR using the RLMRACE Kit (Ambion, Germany) yielded the full length cDNA sequences. To analyze the genomic structure of the genes, different sets of primers were synthesized. Genomic DNA was extracted as described previously (Reski et al., 1994) and used as template for PCR amplification. The resulting PCR products were subcloned in pCR.4-Topo (Invitrogen, Germany) and both strands sequenced with appropriate overlaps by primer walking.

Software

The GCG package 10.2 (Accelrys, U.S.A.) was used for sequence analysis as well as CLUSTAL W 1.81 (Thompson et al., 1994) for multiple sequence alignment. Homology searches were conducted with BLAST 2 (Altschul et al., 1997). Peptide distances were calculated from the alignment using the GCG software Distances with Kimura parameters. The GENPEPT database (release 124.0, www.ncbi.nim.nih.gov), being a conceptual translation of GENBANK, was used as a peptide database covering the known protein coding genes from all organisms. The FtsZ and tubulin motifs were extracted from PROSITE (rel. 16.37, www.expasy.ch).

Database Searches

BLASTP searches against GENPEPT as well as TBLASTN searches against the GENBANK EST_OTHER division (non human/mouse) were run using full-length FtsZ peptide sequences known to belong to the plant FtsZ1 family (Pisum sativum) and FtsZ2 family (Gentiana lutea). From the significant hits a subset of 33 sequences was extracted for further analysis, covering all photosynthetic organisms (cyanobacteria, algae and land plants). Redundant sequences were removed and some ESTs clustered (table 1).

TABLE 1

| organism | nomenclature | family | acc. no. | EST | identical to/cluster with |
|---|---|---|---|---|---|
| Arabidopsis thaliana | 1-1 | 1 | AAA82068 | | BAB08597, cDNA: U39877 gen.: AB010071 |
| Ceratopteris richardii | | 1 | BE643351 | X | |
| Medicago truncatula | | 1 | AW775962 | X | |
| Neottopteris nidus | | 1 | AF275720 | X | |
| Nicotiana tabacum | 1-1 | 1 | CAB89286 | | |
| Nicotiana tabacum | 1-2 | 1 | AAF23770 | | |
| Nicotiana tabacum | 1-3 | 1 | CAB89287 | | |
| Nicotiana tabacum | 1-4 | 1 | CAB41987 | | |
| Oryza sativa | | 1 | AAK64282 | | |
| Physcomitrella patens | 1-1 | 1 | AJ428993 | | |
| Physcomitrella patens | 1-2 | 1 | AJ428994 | | |
| Pisum sativum | | 1 | CAA75603 | | |
| Sorghum propinquum | | 1 | BF588200 | X | |
| Tagetes erecta | | 1 | AAF81220 | | |
| Triticum aestivum | | 1 | BE444075 | X | BE498211 |
| Arabidopsis thaliana | 2-1 | 2 | AAK92779 | | AAC35987, BAB68127, AAD21440 cDNA: AB052757, |
| | | | | | gen.: AC006921 |
| Arabidopsis thaliana | 2-2 | 2 | AAL07180 | | CAB89236, AAK63846, cDNA: AF384167, gen.: AL353912 |
| Chlamydomonas reinhardtii | | 2 | BE441845 | X | BG855721, AV624700 |
| Gentiana lutea | | 2 | AAF23771 | | |
| Lilium longiflorum | | 2 | BAA96782 | | |
| Nicotiana tabacum | 2-1 | 2 | CAB89288 | | |
| Nicotiana tabacum | 2-2 | 2 | CAC44257 | | |
| Physcomitrella patens | 2-1 (“1”) | 2 | CAB54558 | | |
| Physcomitralla patens | 2-2 (“2”) | 2 | CAB76387 | | |
| Anabaena sp. | | cy-ng | AAA85526 | | |
| Cyanidium caldarium | 1 | cy-ng | BAA82871 | | |
| Cyanidium caldarium | 2 | cy-ng | BAA82090 | | |
| Cyanidioschyzon merolae | | cy-ng | BAA85116 | | |
| Guillardia theta | | cy-ng | CAA07676 | | CAB40398 |
| Mallomonas splendens | | cy-ng | AAF35433 | | |
| Nostoc sp. PCC 7120 | | cy-ng | CAA83241 | | |
| Synechococcus sp. PCC 7942 | | cy-ng | AAC26227 | | |
| Synechocystis sp. PCC 6803 | | cy-ng | BAA17496 | | |

Alignment

The above mentioned peptide sequences were subjected to a multiple sequence alignment using default parameters, leading to an alignment of 530 positions. Inspection of the alignment did not reveal any obvious errors, therefore it was not altered before further analyses took place. From the full-length alignment only 330 positions, representing the highly conserved part of the sequences depicted in FIG. 9a/b, were used for phylogenetic analysis, as the divergent N- and C-terminal regions do not allow calculation of an over-all phylogeny using the full-length alignment.

Results and Discussion

Isolation of two New Moss FtsZ Genes, Comparison with Arabidopsis

We cloned and sequenced both the full length cDNA and genomic loci of two novel FtsZ genes, FtsZ 1-1 and 1-2. These new Physcomitrella FtsZ homologues group in the land plant FtsZ1 family and therefore are designated 1-1 and 1-2. The two previously published FtsZ homologues “1” and “2” (Kiessling et al., 2000) cluster in the land plant FtsZ2 dade and are therefore renamed FtsZ 2-1 and FtsZ 2-2, respectively.

The genomic organisation of the Physcomitrella and Arabidopsis FtsZ genes is shown in FIG. 8. Whereas the sequences of FtsZ 2-1 (“1”) and 2-2 (“2”) remarkably resemble each other (Kiessling et al., 2000), FtsZ 1-1 and 1-2 show a different organisation. The genes for 2-1 and 2-2 both contain 6 introns in conserved positions and mainly differ in the slightly different sized exon 1 and intron 6. FtsZ 1-1, on the other hand, contains 5 introns about evenly spaced among the sequence, whereas 1-2 is host to just 3 introns that resemble the positions of intron 2, 3 and 4 of 1-1 or intron 1, 2 and 3 of 2-1/2-2. The close relationship of 2-1 and 2-2 makes it probable that these genes were duplicated only recently, in terms of evolutionary history. Whether or not 1-1 and 1-2 share a recent common ancestor, i.e. evolved by duplication of the gene and subsequent insertion or deletion of introns, cannot be answered yet. Although 2-1 and 2-2 predictably have a low evolutionary distance (17.78 substitutions per 100 positions), they share with 1-1 (86.95/86.98) and 1-2 (93.63/94.81) about the same distance as the latter two amongst each other (92.36). In comparison with the Arabidopsis genes it becomes evident that Arabidopsis 2-1 and 2-2 have a genomic structure resembling those of Physcomitrella 2-1 and 2-2, whereas Arabidopsis 1-1 is remarkably similar to Physcomitrella 1-1.

FIG. 8 also displays a structural feature of the FtsZ proteins: the tubulin/GTPase motif-containing exon (pink). Because of the fundamental importance of this motif for FtsZ function we assume that the exon harbouring the tubulin/GTPase motif is homologous throughout the genes, as is also the case for the two introns bordering this exon. There are in total three introns shared by all the genes, the two abovementioned ones (marked light and dark yellow) as well as another intron 3′ of those, marked in red. Whereas the FtsZ2 family contains an additional intron in the extreme 3′ end (blue, lacking from Arabidopsis 2-1) and between the “dark yellow” and “red” introns (orange), the FtsZ1 family exhibits an intron in the 5′ end of the gene (green, lacking from Physcomitrella 1-2). The intron shown in purple is common to both families as well but lacks in Physcomitrella 1-2 again. In terms of genomic organisation, Physcomitrella 2-1 and 2-2 and Arabidopsis 2-2 strongly resemble each other. The same is the case for the Physcomftrella 1-1 and the Arabidopsis 1-1 genes. The Arabidopsis 2-1 gene, however, lacks one of the family 2 introns. The Physcomitrella 1-2 gene lacks two of the family 1 introns and therefore is quite atypical for its subfamily.

FtsZ Peptide Sequence Features and Patterns

A schematic representation of the FtsZ peptide sequence alignment is shown in FIG. 9a. It shows the location of the PROSITE FtsZ motifs 1 and 2 as well as the Crensing terminal core domain (Ma and Margolin, 1999) and the highly conserved central region of the protein. Both the land plant FtsZ1 and 2 families (according to Osteryoung and McAndrew, 2001; see table 1 for family assignment) as well as the non-green organisms (cyanobacteria and non-green algae) are shown as independent groups. A detailed description of the features present in plant FtsZ sequences can be found in Osteryoung and McAndrew (2001). Here we want to emphasize the differences existing within the three subfamilies, plant FtsZ1 and FtsZ2 as well as cyanobacteria/non-green algae (cy-ng). The existing PROSITE pattern PS01134 is able to find FtsZ proteins from SWISS-PROT with high accuracy (no false positives, low number of false negatives). However, the pattern fails to detect a number of plant FtsZ1/2 family members from GENPEPT. The mismatches in the pattern leading to failure are highlighted in red in FIG. 9c, showing part of our 580 position long FtsZ alignment. The PROSITE pattern PS01135 covers the FtsZ GTP-binding domain that includes the tubulin pattern PS00227 and can detect FtsZ family members in SWISS-PROT with about the same accuracy as the above mentioned pattern. It fails, however, in detecting a number of both FtsZ2 and cy-ng family members because of a mismatch (FIG. 9c). As the region covered by PS01134 allows to distinguish between the subfamilies FtsZ1/2 and cy-ng, we created patterns that enable this task. The pattern FtsZ1-pl: (A,V)(I,V)NTDxQALxx(F,S)ₓ(A,V)x{21,21}(L,T)GE(Q,E) is able to find all FtsZ1 family members in GENPEPT (without false positives/negatives), as is the case for the FtsZ2-pl called pattern (V,A)NTD(I,V,A)QA(M,I,L)(R,K,A)xSPVx{23,23}IG(M,C,A)(N,S,K) for FtsZ2 and (I,V,L)NTD(A,S,V)QALxxxx(A,T)x{17,17}G(N,K)P(A,E)(I,V)Gx(K,Q) for cy-ng (for details see FIG. 9c); these new patterns are shown in FIG. 9b. These patterns will allow to scan for new FtsZ sequences belonging to those groups in further releases of the international databases.

### REFERENCES

Kiessling, J., S. Kruse, S. A. Rensing, K. Harter, E. L. Decker, and R. Reski. 2000. Visualization of a cytoskeleton-like FtsZ network in chloroplasts. J. Cell. Biol. 151:945-950.
Ma, X., and W. Margolin. 1999 Genetic and functional analyses of the conserved Cterminal core domain of Eschenchia coli FtsZ. J. Bacteriol. 181:7531-7544.
Osteryoung, K. W., and R. S. McAndrew. 2001. The plastid division machine. Annu. Rev. Plant. Physiol. Plant. Mol. Biol. 52:315-333.
Rensing, S. A., S. Rombauts, Y. Van de Peer, and R. Reski 2002. Moss transcriptome and beyond. Trends. Plant Sci., in press
Thompson, J. D., D. G. Higgins, and T. J. Gibson 1994. CLUSTAL W: improving the sensitivity of progressive multiple sequence alignment through sequence weighting, position specific gap penalties and weight matrix choice. Nucl. Acids. Res. 22:4673-4680.

### Example 2

### Localisation of FtsZ1-2 in Plant Cells

Here we report on a Physcomitrella homolog from the FtsZ1 family. In transiently transfected moss protoplasts PpFtsZ1-2::GFP fusion proteins assemble to ring-like structures not only in plastids but also in the cytoplasm (FIG. 6a), indicating a dual targeting of PpFtsZ1-2 and an involvement of this specific protein not only in plastid but also in cell division.

Dual targeting of nuclear-encoded proteins to different cell compartments normally requires two different translation initiation sites in the 3′ end of the messenger RNA. The N-terminus of PpFtsZ1-2 harbors two methionine residues at positions 1 and 108, respectively, indicating such dual targeting.

The first 107 amino acid residues of this protein were sufficient to target a GFP-protein exclusively to the plastids of transiently transfected moss protoplast where the protein was evenly distributed in the stroma (FIG. 7a). In contrast, a truncated PpFtsZ1-2::GFP fusion protein starting at the second methionine (aa residue 108) was exclusively located in the cytoplasm where it polymerized to filamentous, ring-like structures (FIG. 6b). When this second methionine was mutated, a full-length PpFtsZ1-2::GFP fusion protein was exclusively targeted to the plastids where it assembled to filamentous, ring-like structures (FIG. 6c).

From this it was concluded, that the second in-frame ATG was the initiation site for the cytosolic version of this novel Physcomitrella FtsZ (PpFtsZ1-2) and that polymerization to filaments is a characteristic inherent to the mature FtsZ protein and most likely not an artifact due to over-expression.

As a further control an FtsZ gene from the second family (PpFtsZ2-1) was analyzed. Normally, GFP fusions with this protein are exclusively found in the plastids where they polymerize to filamentous networks. However, this gene harbors a second in-frame ATG at a similar position in the 5′ end as PpftsZ1-2 does. A truncated FtsZ2-1 starting with this additional methionine resulted in dot-like aggregates in the cytosol (FIG. 6d) while GFP alone is evenly distributed within the cytosol of transiently transfected moss protoplasts (FIG. 7b).

This indicates that PpFtsZ2-1 needs co-factors for assembly, which are only present in plastids. In contrast, the novel PpFtsZ1-2 described here, obviously finds polymerization-stimulating co-factors in plastids as well as in the cytosol, suggesting a distinct biological function of this ancient tubulin in both cell compartments.

### Example 3

### Immunocytochemistry of Endogenous FtsZ1-2 in Physcomitrella Protonema, a Moss Cell Filament

For visualizing endogenous FtsZ in non-transgenic moss cells by immunocytochemistry, a polyclonal antibody was raised against the C-terminal aa 441-490 of FtsZ1-2 where similarity with other FtsZ homologs is low (−27%) and even lower with tubulins. In the protein extracts of Physcomitrella, the anti-FtsZ-antibody detected specific bands of approximately 40 kD and 46/47 kD, respectively (FIG. 10A). The sizes of the bands support the findings on dual localization of the FtsZ1-2: the predicted masses for the cytosolic (40.5 kD) and the plastidic (44.6 kD) form correspond well with the sizes of the bands detected via immunoblotting. In addition, the 46/47 kD double band may hint to posttranslational modifications and a faint band of approximately 75 kD to the dimerization of cytosolic FtsZ1-2. Cross-reactivity was excluded by use of a specific anti-tubulin antibody which detected proteins of −51 kD (FIG. 10 A).

In immunocytochemistry this anti-FtsZ-antibody labeled not only the division site of chloroplasts (FIG. 10G) but additionally visualized a cytoplasmic signal allocating cytosolic localization of the FtsZ1-2 protein (FIG. 10B-F). At the emerging cell plate between dividing cells, prominent annular to discoidal structures were observed (FIG. 10B-F) that resembled the phragmoplast, a microtubular structure guiding the formation of cell plates during late phases of mitosis in plants, including Physcomitrella. Thus, the first evidence for a cytosolic localization of an FtsZ1-2 in a eukaryote is provided.

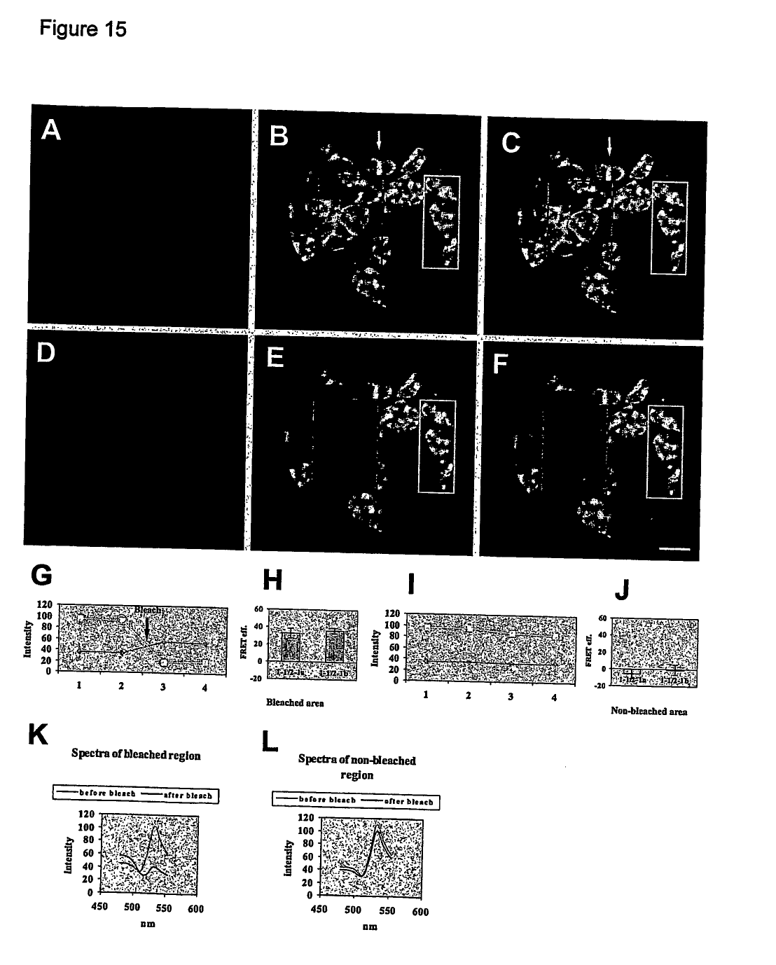

In order to test whether FtsZ1-2 colocalizes with tubulin, double-labeling of FtsZ1-2 together with tubulin was performed using non-tranfected plant cells. By double-channel confocal microscopy, these cells were analysed. Prior to mitosis, FtsZ1-2 was detected in an equatorial band surrounding the nucleus which seemed to be tethered to this band through filamentous structures contiguous with the nuclear envelope (FIG. 10H). Comparison with tubulin signals (FIG. 101) identified these FtsZ1-2 structures as part of the preprophase band, a plant-specific microtubular structure, which is formed before the onset of mitosis predicting the future division site. Thus, the FtsZ1-2 may function not only in organelle but also in cell division.

### Example 4

### The Role of FtsZ1-2 in Cell Division

For assessing the role of FtsZ1-2 in cell division, transient transfection experiments were carried out. These transfection experiments demonstrate that a high level of cytosolic FtsZ1-2-GFP impaired cell division (FIG. 11A,B). Whereas non-transfected Physcomitrella cells managed to divide at the latest nine days after isolation, cells expressing high amounts of FtsZ-1-2-GFP (which is dually targeted to chloroplasts and to the cytosol) were severly affected in cell division: more than 30% of all transfected cells were impeded in cell division revealing ring-like structures at the future division site (FIG. 11A, B). By comparison, protoplasts harboring only low FtsZ-1-2-GFP levels managed to divide but demonstrated a block plastid division: they contained only one or a few huge plastids rather than about 50 small, wild-type-like chloroplasts (FIG. 11C).

In order to more precisely allocate function to localization of the protein, effects of the mutated FtsZ-1-2-GFP proteins on cell division were monitored. High levels of the cytosolic fusion protein (for localization refer to FIG. 6b) also impeded cell division (which represented more than 30% of all transfected cells (FIG. 11A) whereas high levels of the plasitidic fusion protein (for localization refer to FIG. 6c) only blocked organelle division (data not shown) while cell division apparently was not affected (FIG. 11A). As a further control moss cells were analyzed which harbored FtsZ2-1-GFP artificially targeted to the cytosol (for localization refer to FIG. 6d). Some of these cells containing high amounts of the accumulated fusion protein were also impeded in cell division (FIG. 11A). However, the effects of the originally plastidic isoform were significantly different from the effects of the cytosolic FtsZ isoform: FtsZ2-1 was not able to polymerize to filaments in the cytosol (FIG. 6d), and, moreover, had less severe effects on cell division than the dually targeted FtsZ1-2 (FIG. 11A), indicating that both isoforms recruit additional proteins from the cytoplasm at different efficiencies in vivo.

### Example 5

### Localization of FtsZ1-1 in Plant Cells

Here a further Physcomitrella homolog from the FtsZ1 family is studied. After transient transfection of protoplasts PpFtsZ1-1-GFP fusion proteins were detected inside plastids assembling into filamentous networks there (FIG. 12A-D). In addition, FtsZ1-1 filaments were observed that seemed to connect the chloroplasts with each other (FIG. 12A,B, arrows). Moreover, in some protoplasts, full-length FtsZ1-1 polymerized not only into network-like structures but also assembled into a ring inside plastids (FIG. 12C,D).

In order to further analyze the localization of PpFtsZ1-1, different GFP fusions were created. An N-terminal part of FtsZ1-1 (amino acid (aa) 1-84) fused to GFP was localized exclusively inside plastids (FIG. 12E,F) consistent with the predicted cleavage site at aa 33 obtained via in silico predicition using TargetP (Nielsen et al., 1997, Emanuelsson et al., 2000). Thus, this part of the protein contains a functional transit peptide. A truncated version of FtsZ1-1 starting with methionine 85 was artifically targeted to the cytosol where it assembled into network-like structures (FIG. 12G,H).

Although some PpFtsZ isoforms exhibit different subcellular localization patterns in moss protoplasts, they act similarly on plastid division: A slightly elevated level of each distinct isoform, for example FtsZ1-1 (FIG. 13A,B) or FtsZ1-2 (FIG. 13C,D), (indicated by faint GFP fluorescence) led to “minichloroplasts” that are significantly smaller than wild type chloroplasts whereas a high level of each isoform, for example FtsZ1-1 (FIG. 13E,F) or FtsZ1-2 (FIG. 11C), (indicated by strong GFP fluorescence) completely blocked plastid division.

### Example 6

### FRET Analysis Reveals a Direct Protein-Protein Interaction Between FtsZ1-1-YFP and FtsZ2-1-CFP

For FRET (fluorescence resonance energy transfer) analysis, the full length cDNAs of the different PpftsZ homologs were subcloned into two different expression vectors coding for CFP or YFP, respectively. Physcomitrella protoplasts were transiently transfected with pairs of these constructs and FRET was analyzed via a confocal microscope two or three days after transfection. Different types of positive and negative controls were performed to make sure that true positive signals were obtained. On the one hand, a CFP-YFP fusion served as a positive control in which both proteins are connected by two amino acids. This control revealed FRET due to the close neighbourhood of CFP to YFP (FIG. 14A). Thus, according to this positive control, FRET could be detected via the system used.

In previous studies PpFtsZ-GFP fusions assembled into filamentous formations which indicated self-interactions of the corresponding isoforms. Thus, these putative protein-protein interactions served as further “positive controls”: YFP and CFP fusions of a single PpftsZ homolog were cotransfected and FRET was detected due to the self-interaction. Like for the first positive control, high FRET efficiencies were obtained for these experiments (FIG. 14B-E).

In addition, two types of negative controls were performed: On the one hand, lack of FRET between a FtsZ1-2-CFP fusion (donor) and YFP (“acceptor”) (FIG. 14L) demonstrated that FRET is not due to an interaction between CFP and YFP, but a consequence of an interaction between the two proteins of interest. The second negative control—a non-bleached area compared to the bleached region within the same cell—also resulted in negative or very low FRET efficiencies (FIG. 14M-P).

Finally, to analyse an interaction between members of both FtsZ families, protoplasts were cotransfected with PpftsZ1-1-yfp and PpftsZ2-1-cfp. Confocal laser scanning microscopy demonstrated that the fusion proteins polymerized into some ring-like structures inside plastids (FIG. 15, arrows) apart from additional aggregations (FIG. 15). Moreover, FRET analysis of these protoplasts revealed a direct interaction of both FtsZ isoforms: Bleaching of a defined region (FIG. 15, red square) resulted in a high increase of CFP fluorescence in this area (FIG. 15F, red square) whereas in a non-bleached region, CFP intensity slightly decreased due to the imaging process (FIG. 15F, green square). Acceptor bleaching of this defined region revealed a FRET efficiency of 36.5% (FIG. 15H) (whereas the non-bleached control area exhibited a negative value (FIG. 15J)). Compared to other values reported in recent FRET studies, this FRET efficiency is rather high. Thus, a direct interaction of PpFtsZ1-1 and PpFtsZ2-1 could be detected in vivo.

1. A nucleic acid containing a nucleotide sequence encoding a polypeptide having an amino acid sequence selected from the group consisting of SEQ ID No. 3, SEQ ID No. 4 and SEQ ID No. 5, or a biologically active derivative thereof, said polypeptide or biologically active derivative thereof being a target for herbicidal compounds in plants.

2. The nucleic acid according to claim 1, wherein said nucleotide sequence is selected from the group consisting of SEQ ID No. 1 and SEQ ID No. 2.

3. A vector containing the nucleic acid according to claim 1.

4. A host organism containing the nucleic acid according to claim 1.

5. A transgenic plant containing the nucleic acid according to claim 1.

6. A polypeptide having an amino acid sequence selected from the group consisting of SEQ ID No. 3, SEQ ID No. 4 and SEQ ID No. 5, or a biologically active derivative thereof, said polypeptide or biologically active derivative thereof being a target for herbicidal compounds in plants.

7. A method for developing herbicidal compounds, comprising the steps of:
contacting a test system containing a polypeptide having an amino acid sequence selected from the group consisting of SEQ ID No. 3, SEQ ID No. 4 and SEQ ID No. 5, or a biologically active derivative thereof, with a candidate compound to be assayed; and
measuring the herbicidal activity of said candidate compound.

8. A herbidical compound obtained by using the method according to claim 7.

9. A vector containing the nucleic acid according to claim 2.

10. A host organism containing the nucleic acid according to claim 2.

11. A host organism containing the vector according to claim 3.

12. A transgenic plant containing the nucleic acid according to claim 2.

13. A transgenic plant containing the vector according to claim 3.
